(19) United States
(12) Patent Application Publication
(10) Pub. No.: US 2005/0283228 A1
(43) Pub. Date: Dec. 22, 2005

(54) Expandable stent with relief holes capable of carrying medicines and other materials

(76) Inventor: Ulf Harry Stanford, Incline Village, NV (US)

Correspondence Address: Bruce H. Johnsonbaugh;Eckhoff & Hoppe, 333 Sacramento Street, San Francisco, CA, 94111, US

(21) Appl. No.: 11/213,132

(22) Filed: Aug. 26, 2005

Related U.S. Application Data
(63) Continuation-in-part of application No. 10/695,130, filed on Oct. 28, 2003.
(63) Continuation-in-part of application No. 10/000,533, filed on Oct. 30, 2001, now abandoned.
Application No. 10/000,533 is a continuation-in-part of application No. 09/774,760, filed on Jan. 30, 2001, now abandoned.
Application No. 10/000,533 is a continuation-in-part of application No. 09/357,699, filed on Jul. 20, 1999, now abandoned.
(60) Provisional application No. 60/094,540, filed on Jul. 29, 1998.

(51) Int. Cl. A61F 2/06

(52) U.S. Cl. 623/1.15

(57) ABSTRACT

An array of arcuate relief holes is formed at the flexion points formed at the junctures of two or more struts of an expandable stent. The relief holes are small enough to preserve the columnar compressive strength of the stent as well as the resistance of the struts to twisting and warping. The relief holes allow a balloon expandable stent to be expanded with less balloon pressure. The relief holes allow the use of wider and thinner material in the stent, giving greater vessel coverage while simultaneously reducing the stent profile and increasing laminar blood flow through the stented vessel. The struts have a cross-sectional width that remains constant at the junctures or flexion points, minimizing or eliminating plastic deformation of the struts at the flexion points and between flexion points. Patterns of relief holes may be placed in stents to achieve controlled, non-uniform expansion and which, in some instances, allow the stent to bend easily in a given direction to facilitate deployment in curved arteries or other body lumens. Medicinal or other coatings may be applied to the stent and into the relief holes to increase the adhesion of the coating to the stent.

### CROSS REFERENCE TO RELATED APPLICATIONS

This is a continuation-in-part of U.S. patent application Ser. No. 10/695,130 filed Oct. 28, 2003 and entitled EXPANDABLE STENT WITH ARRAY OF RELIEF CUTS, and is a continuation-in-part of U.S. patent application Ser. No. 10/000,533 filed Oct. 30, 2001; both of which were continuations-in-part of U.S. patent application Ser. No. 09/774,760 filed Jan. 30, 2001 and entitled EXPANDABLE STENT WITH ARRAY OF RELIEF CUTS, and which was a continuation-in-part of U.S. patent application Ser. No. 09/357,699 filed Jul. 20,1999 and entitled EXPANDABLE STENT and claims the benefit of U.S. provisional application Ser. No. 60/094,540 filed Jul. 29, 1998 entitled EXPANDABLE STENT.

### BACKGROUND AND BRIEF SUMMARY OF THE INVENTION

The present invention relates generally to balloon expandable and self-expanding stents made of solid, non-porous and non-tubular material such as nitinol, stainless steel, cobalt chromium, plastic or plastic degradable material. More particularly, the present invention provides an array of arcuate “relief holes” for use in a variety of stent designs. According to the present invention, the relief holes are typically cut with lasers and strategically placed along the centerline of a strut and at or near a juncture or flexion point formed at an intersection of two or more struts (without reducing the width of the strut at the flexion point) to allow the stent to expand more easily, requiring less pressure for the expansion, and optimizing the strength of each strut and its resistance to bending, warping and fatigue. By limiting the size of the relief holes, and by maintaining a constant width of the struts through the flexion points, any plastic deformation is limited or eliminated. Prior art stent designs such as Shanley U.S. Pat. No. 6,293,967 rely on plastic deformation at the flexion points as the stent expands. Plastic deformation is achieved by Shanley by reducing the strut width at flexion points. The plastic deformation at flexion points weakens the stent and allows twisting, warping and buckling at the flexion points. The present invention is the direct opposite of Shanley, in that the present invention minimizes or eliminates plastic deformation as the stent expands. The stents utilizing the present invention have a designed range of expansion, for example a 50% range, and within that designed range, the flexing and bending of the struts is elastic only, i.e., no plastic deformation within the designed range of expansion. The purpose of the relief holes is to allow the stent (or a portion of the stent) to expand more easily, while maximizing elastic flexing and bending of the material at flexion points and minimizing or eliminating any plastic deformation at the flexion points. The present invention allows the use of wider and thinner material for the struts, resulting in a stent with increased surface area for carrying medicine, and resulting in a thinner walled stent allowing maximum blood flow through the artery or other lumen.

The prior art also includes the Thompson U.S. Pat. No. 6,132,461. That patent discloses a “double-strut” stent configuration with a plurality of closed cells wherein each member of the cell configuration is slotted throughout its entire length. The inherent weakness of this design is that each fully slotted cell member becomes significantly weakened by the use of slots extending throughout its entire length. The slotted member will deform plastically if slotted at a flexion point. Also, the compressive strength of each member is substantially weakened as shown by the “critical load” analysis as an “Euler column” as established mathematically by Leonard Euler. The removal of approximately one-third of the material along the center of a column and throughout the entire length of the column greatly increases its tendency to deform in a “plastic” fashion, and reduces its ability to support a compressive load, i.e., its hoop strength in the case of a stent. In contrast to the “double-strut” design described in the above-identified Thompson patent, the present invention utilizes an array of strategically placed relief holes. Each individual relief hole has a relatively short length in order to preserve the ability of the member to retain its columnar compressive strength and its resistance to plastic deformation.

The prior art stent designs typically use stent members or struts having circular or rectangular cross sections. These prior art struts have several disadvantages. First, as the stents are downsized for use in smaller vessels, the circular or rectangular cross sections of the stent material tends to effectively reduce the cross-sectional area of the artery or other body lumen which is capable of achieving low turbulence or laminar blood flow. A second disadvantage is limited coverage of the vessel wall by the stent. Third, and perhaps most important, is the disadvantage that downsized prior art struts have a smaller surface area for carrying medicine. The present invention is designed to overcome these problems by providing a wider and thinner material, enhanced vessel wall coverage and enhanced surface area for carrying medicine, but at the same time providing a stent that will expand as easily as the prior art stent.

It is also known in the prior art to electro-polish portions of a stent to reduce the cross-sectional area of a stent member so that less balloon pressure is required to expand the stent. The electro-polishing technique is very expensive and has the inherent weakness of reducing the resistance of the stent to twisting and warping. The present invention avoids the cost and disadvantages of electro-polishing; furthermore, the present invention inherently allows the use of wider and thinner stent struts or members.

A significant feature of the invention is that the stent is capable of carrying medicines (and other materials) for use in blood vessels, the urethra and other body lumens. More particularly, the present invention provides one or more relief holes formed in a stent to either carry a “plug” of medicine (for example) within each relief hole or to increase the adhesion of a medicinal coating (for example) applied to the surface of the stent. The present invention allows stents to carry various materials, including medicines, lubricants, chemicals and radioactive materials. The present invention in its preferred form allows the use of wider and thinner struts, while simultaneously providing the presence of relief holes to increase adhesion of medicinal coatings. The wider and thinner struts provided by this invention are very resistant to twisting or warping, since each strut retains its width at its flexion points. In contrast, some prior art stent designs increase flexibility of the stent by significantly reducing the strut width at flexion points; significant disadvantages of the prior art approach are increased cost, increased tendency of the struts to twist or warp as the stent expands, and possible puncturing of the artery or balloon as the struts twist and/or warp.

The present invention also facilitates the use of multiple layers of different types of medicines or combinations of different materials in a multi-layered coating. Alternatively, different regions of the stent surface may be coated with different materials.

The relief holes of the present invention are sufficiently small so that the structural strength of each strut between flexion points is not significantly reduced, as compared with the same strut without relief holes. The word “strut” is used broadly herein, and is used to refer to one of a series of interconnected members wherein those interconnected members flex at flexion points as the stent expands. Although the present invention uses relief holes at flexion points to allow stents to expand with less pressure, the strength of each strut or interconnected member between flexion points is not significantly reduced. That is, each strut (or inter-connected member) does not significantly lose its resistance to bending, twisting or buckling between flexion points because of the present of relief holes according to the invention.

Another advantage of the present invention is that the relief holes may be applied together with coatings to a variety of existing and commercially successful balloon expandable and self-expanding stent designs. The use of relief holes as described and claimed herein can quickly provide existing commercial stents with most of the advantages of the present invention.

Another significant problem with most prior art stent designs arises when a stent is placed in a curved section of an artery (or other lumen). As the balloon is expanded, the stent tends to straighten, causing the curved portion of the artery (or other lumen) to straighten and sometimes rupturing the vessel wall. The present invention facilitates expansion of a stent in a curved artery by using curved balloons and applying relief holes in selected, predetermined patterns to the stent. The relief holes reduce the tendency of the stent to straighten as the stent and balloon are expanded.

A further limitation of prior art stents is that the typical stent expands at a uniform rate as balloon pressure is applied. There are many practical instances where a controlled non-uniform expansion of a stent would be a significant advantage.

Another significant aspect of the present invention is that selective placement of an array of relief holes on a stent allows the stent to expand in a predetermined and controlled non-uniform fashion. For example, placing an array of relief holes only in the longitudinal center region of the stent causes the center region of the stent to expand first before the distal and proximal regions of the stent expand. As another example, relief holes can be utilized only at the distal and proximal end regions of the stent, which causes the end regions to expand first, capturing multiple embolic particles, with the central region of the stent expanding last. As a further example, relief holes may be applied in various patterns to cause stents to act differently; some patterns allow stents to be used better in curved and tapered vessels, some patterns allow one or both ends of the stent to be “flared,” and some patterns allow the stent to bend more easily in a given direction.

Another advantage of the present invention is that the relief holes may be applied to a variety of existing and commercially successful balloon expandable and self-expanding stent designs. The use of the relief holes, as described and claimed herein, can quickly provide existing commercial stents with most, if not all, of the advantages of the present invention.

It is therefore a primary object of the present invention to provide an array of arcuate relief holes in a balloon expandable or self-expandable stent to allow the stent to expand more easily and with less pressure, without any significant loss of the strength or resistance to twisting or warping of the stent members in which the holes are formed.

Another object of the invention is to provide an array of arcuate relief holes in prior art stent designs to allow those stents to expand more easily and with less pressure than is the case in the absence of relief holes.

Still another object of the invention is to provide a balloon expandable and self-expandable stent design having an array of arcuate relief holes, which in turn allows the use of wider and thinner members in the stent to increase the vessel wall coverage of the stent, while using thinner wall members.

Still a further object of the invention is to provide an array of arcuate relief holes which, not only allows the use of wider members, but also allows the use of thinner wall stents, thereby increasing the effective inner diameter of arteries and other lumens carrying those stents. The use of thinner walled stents minimizes the profile or cross section of the stent and provides more clearance in inserting and deploying the stent.

A still further object of the invention is to provide selective placement of one or more arrays of arcuate relief holes to a stent, which allows the stent to expand in a predetermined and controlled non-uniform fashion. This feature allows a stent to be custom designed to an artery to better support the arterial wall and to seal end leaks.

A further object of the present invention is to provide one or more relief holes in an expandable stent to increase the adhesion of a medicinal or other coating applied to the stent.

Another object is to provide a stent having relief holes and being coated with multiple layers of different materials, or to apply different coatings to several regions of a single stent.

Still another object of the invention is to provide a balloon expandable and self-expandable stent design having an array of “flexion” relief holes which not only increase adhesion of coatings, but also allows the use of wider and thinner members in the stent to increase the vessel wall coverage of the stent.

A still further object of the invention is to provide one or more relief holes to a stent, wherein each relief hole carries a “plug” of medicine or other material.

Another object is to provide a coated stent with relief holes, wherein the surface coating dissolves into the vessel wall and thereafter the “plugs” of material carried within the relief holes dissolve into the vessel wall.

Other objects and advantages of the present invention will become apparent from the following description and the drawings wherein:

### BRIEF DESCRIPTION OF THE DRAWINGS

FIG. 1A illustrates a conventional, prior art diamond-shaped stent cell;

FIG. 1B illustrates relief holes of the present invention applied to the stent of FIG. 1A;

FIG. 1C illustrates how relief holes of the present invention with a non-circular shape are applied to the prior art stent of FIG. 1A;

FIG. 2A illustrates the application of the relief holes of the present invention to a stent cell wherein the struts have a greater width and smaller thickness than the prior art cell of FIG. 1A;

FIG. 2B is a perspective view of a portion of FIG. 2A

FIG. 2C illustrates an alternate pattern of relief holes used at or near a flexion point;

FIG. 2D illustrates yet another pattern of relief holes used at or near a flexion point;

FIG. 2E shows an alternate design of a relief hole for use at or near a flexion point;

FIG. 3A illustrates a prior art six-sided stent cell configuration;

FIG. 3B illustrates the application of the present invention to a prior art cell configuration of FIG. 3A;

FIG. 3C illustrates a six-sided stent cell configuration similar to that shown in FIGS. 3A and 3B wherein the width of the struts has been increased and the thickness decreased;

FIG. 3D illustrates the stent cell of FIG. 3C to which a medicinal coating has been applied;

FIGS. 4A and 4B illustrate how the invention with thinner and wider struts increases the capacity of a given artery for laminar, unobstructed blood flow;

FIGS. 5A-5D illustrate how the relief holes of the present invention may be applied strategically to the distal and proximal ends of the stent, thereby causing those ends of the stent to expand before the central portion of the stent expands;

FIGS. 6A-6C are schematic representations illustrating how a pattern of relief holes placed only in the central portion of the stent would cause that center portion of the stent to expand before the end portions;

FIG. 7 is a schematic representation showing how the relief holes of the present invention can be applied to only the central portion of a prior art stent configuration to cause a predetermined, controlled, uneven expansion of the stent;

FIGS. 8A and 8B are schematic illustrations representing how the present invention could be utilized to seal off the blood flow to and kill a cancerous tumor;

FIG. 9 is a schematic representation showing how relief holes of the present invention may be used in a prior art stent having a single strand serpentine-shaped design;

FIG. 10 is a schematic representation of an alternate stent design in which the invention may be utilized;

FIG. 11A illustrates schematically how relief holes may be patterned for use on a stent to be placed in a curved artery or other body lumen wherein no relief holes are placed in the central region of the stent;

FIG. 11B is a section on the line 11B-11B of FIG. 11A;

FIG. 12A is a schematic representation showing still another way in which relief holes may be patterned on a stent for use in a curved artery or other body lumen;

FIG. 12B is a sectional view on the line 12B-12B of FIG. 12A;

FIG. 13A shows another embodiment wherein relief holes are placed only in the central region of a stent and around the entire periphery of the stent for use in a curved artery;

FIG. 13B is a sectional view on the line 13B-13B of FIG. 13A;

FIG. 14A is yet another embodiment showing relief holes applied only to the central region of the stent for use in a curved artery wherein the relief holes are placed away from the inside radius of curvature of the artery;

FIG. 14B is a section on the line 14B-14B of FIG. 14A;

FIGS. 15A and 15B are schematic illustrations showing how the present invention may be used with bifurcated stents;

FIGS. 16A and 16B are schematic illustrations showing how the present invention may be used in an irregularly shaped artery (or other lumen);

FIGS. 17A and 17B are schematic illustrations showing one way in which the present invention may be utilized in an artery (or other lumen) having a branch artery (or lumen) at or near the deployment site of the stent;

FIGS. 18A and 18B are schematic illustrations showing a second way in which the present invention may be utilized in an artery (or other lumen) having a branch artery (or lumen) at or near the deployment site of the stent;

FIG. 19 is a section on the line 19-19 of FIG. 3D; and

FIG. 20 is an alternate embodiment of that shown in FIG. 19.

### DETAILED DESCRIPTION OF THE DRAWINGS

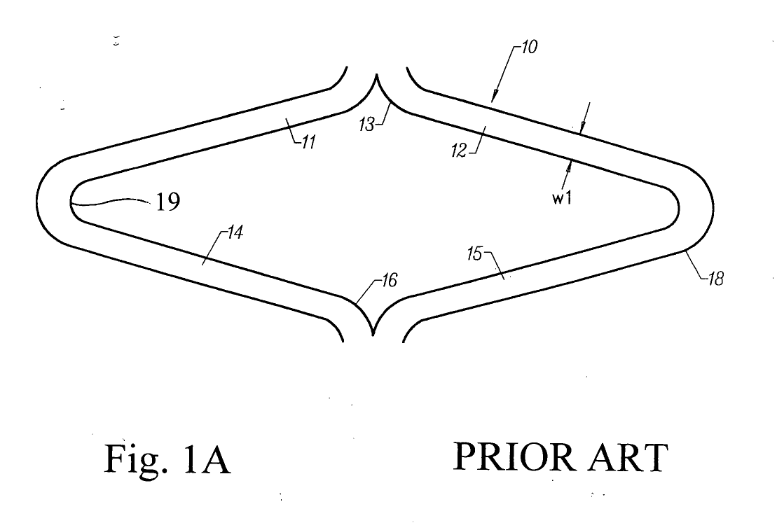

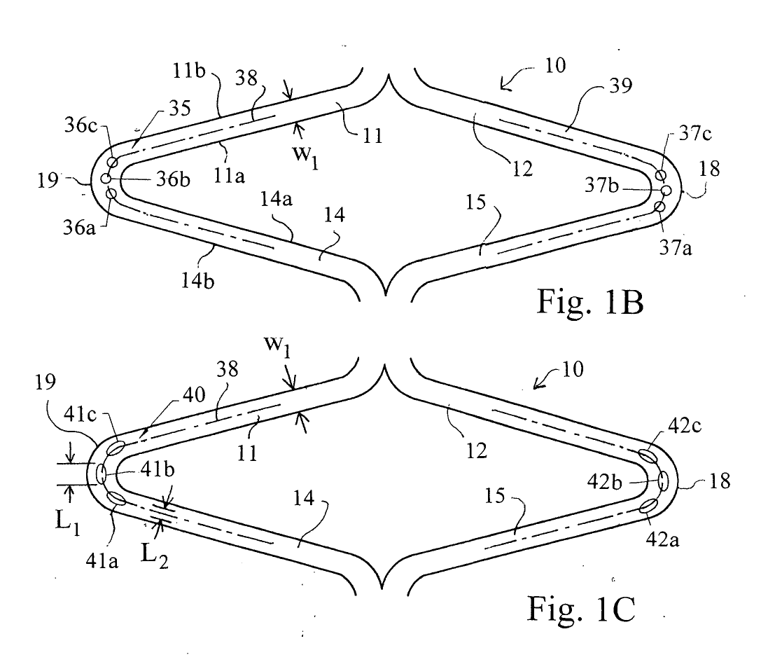

FIG. 1A illustrates a conventional, prior art diamond-shaped stent cell 10. Struts or members 11 and 12 are connected at their intersection 13 and members or struts 14 and 15 are connected at their intersection 16. As the stent cell 10 expands, struts 12 and 15 flex or bend relative to each other at their connection point 18, which is also referred to herein as an intersection point, juncture or a flexion point. Similarly, members 11 and 14 bend or flex relative to each other about their intersection, juncture or flexion point 19.

FIG. 1B illustrates how the arcuate relief cuts or arcuate relief holes of the present invention are applied to the prior art stent 10, shown in FIG. 1A. The relief cuts or relief holes are shown generally as 35 in FIG. 1B and include an array of three holes or passageways 36a, 36b and 36c formed at or near flexion point 19 of struts 11 and 14. Similarly, an array of three relief holes or passageways 37a, 37b and 37c is formed at or near flexion point 18 between struts 12 and 15. Each of the arcuate “relief holes” is an enclosed passageway (i.e., a passageway with boundaries formed within a strut) that extends completely through the strut and is formed along the center line 38 of struts 11 and 14 and the center line 39 of struts 12 and 15 and equidistantly from the edges of the strut. It is significant that each of the relief holes is formed between the edges 11a and 11b of strut 11 and edges 14a and 14b of strut 14. By placement of the relief holes equidistantly away from the strut edges and along the center line of the strut, no material is removed from the edge of the strut at the flexion point, i.e., the width of each strut at the flexion point remains as wide or wider than the width of the strut between flexion points. This is in contrast to the teaching of Shanley U.S. Pat. No. 6,293,967, which reduces the width of struts at flexion points. Maintaining the width of the strut at the flexion point increases the resistance of struts 11 and 14 to twisting and warping as the stent expands. Each of the circular relief holes in FIG. 1B has a diameter that is between 30% and 70% of the overall width of the strut. The relief holes may have different diameters; for example, relief holes 36b and 37b (located at the flexion point) may have larger diameters than relief holes 36a, 36c, 37a and 37c. The relief holes are preferably formed by laser cutting. The relief holes of the present invention are utilized in stents having solid struts as noted above.

FIG. 1C shows an alternate form of the invention wherein an array of elliptical or elongated relief holes 41a, 41b and 41c is formed at or near flexion point 19 of struts 11 and 14 and an array of elliptical relief holes 42a, 42b and 42c is formed at or near flexion point 18 of struts 12 and 15. Each of the elliptical relief holes is placed on the center line 38 of struts 11 and 14. Each of the relief holes is an enclosed passageway that extends entirely through the strut. The transverse dimensions (i.e. perpendicular to center line 38) of each of the elliptical relief holes are between 30% and 70% of the width of strut 11 or 14. That is the width L₂ of each hole is between 30% and 70% of the strut width w₁. The length L₁ of each hole (i.e. the distance along center line 38) is less than the width of the strut. Limiting the size of each relief hole in this fashion minimizes plastic deformation as the stent expands. The relief holes may have an arcuate shape other than circular or elliptical. The term “arcuate” is used herein and in the claims in a broad sense to include circular, oval, elliptical and other smooth, curved shapes having no corners.

It is significant to note that FIGS. 1B and 1C illustrate the present invention as applied to a prior art stent cell configuration 10. The width w₁ of each strut is typically the same as the thickness of each strut, since the struts are typically circular or square in cross section.

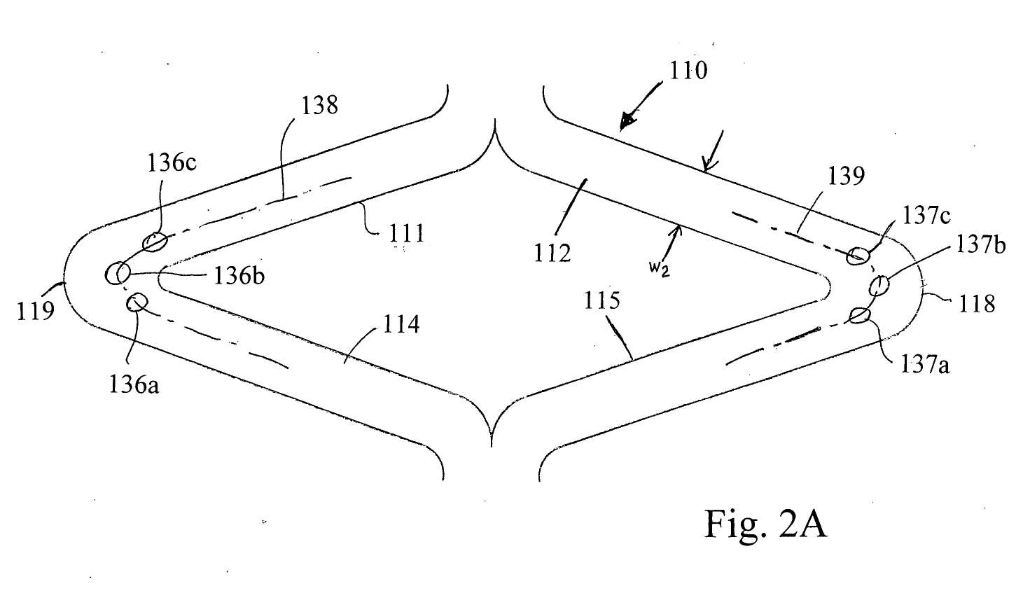

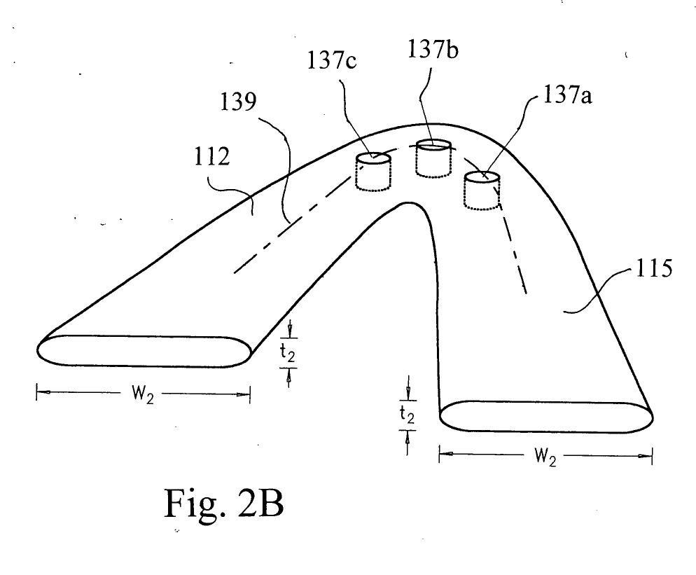

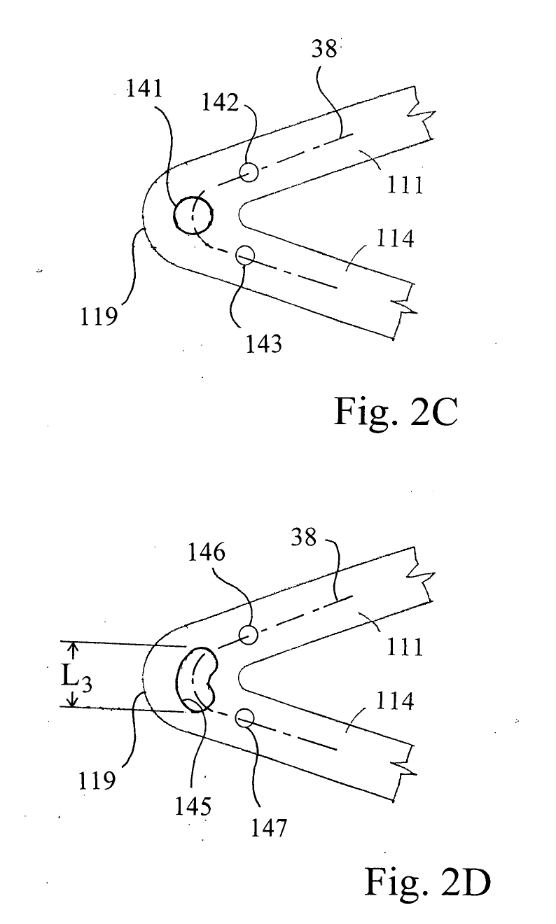

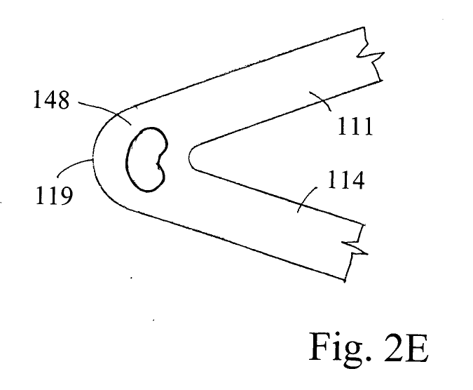

FIGS. 2A and 2B show how the relief holes of the present invention can be applied to a stent cell configuration wherein the struts have a width W₂ which is significantly greater than the width w₁ of the prior art stent cell configuration shown in FIGS. 1B and 1C. Furthermore, the improved stent configuration shown in FIGS. 2A and 2B has a reduced thickness t₂ which may be significantly less than the thickness of the struts of the prior art configuration shown in FIGS. 1B and 1C.

As shown in FIG. 2A, stent cell 110 includes four struts, 111, 112, 114 and 115. Each of the struts 111, 112, 114 and 115 has a width w₂ and a thickness t₂ (FIG. 2B). The width w₂ is between 1.5 and five times greater than the thickness t_{2.} An array of three circular relief holes 136a, 136b and 136c is formed at or near the intersection or flexion point of struts 111 and 114. Each of the relief holes 136a-136c is centered on center line 138 of struts 111 and 114. An array of relief holes 137a, 137b and 137c is formed in struts 112 and 115 at or near their intersection or flexion point 118. Each of the six relief holes shown in FIG. 2A is circular, extends completely through the strut from top to bottom and has a diameter that is between 30% and 70% of the width w₂ of the struts.

FIG. 2B is a perspective view of a portion of FIG. 2A, showing relief holes 137a, 137b and 137c extending through the struts 112 and 115, and formed on center line 139 of struts 112 and 115.

FIGS. 2C and 2D illustrate alternate patterns of relief holes used at and/or near flexion point 119 of struts 111 and 114. FIG. 2C shows a relatively large diameter, circular relief hole 141 located at flexion point 119 and two smaller, circular relief holes 142 and 143 located near flexion point 119. FIG. 2D shows a relatively large, oval shaped relief cut 145 (which may be non-elliptical) located at flexion point 119, and two circular relief holes 146 and 147 located near flexion point 119. Relief cut 145 has a transverse dimension (i.e. perpendicular to center line 38 that is between 30%-70% of the width of the strut). Relief cut 145 has a longitudinal dimension L₃ (i.e. along the center line 38) that is less than the width of the strut.

FIG. 2E shows a further alternate wherein a single, arcuate relief hole 148 is located at flexion point 119.

The relief holes are preferably circular, but may be any arcuate or ovoid shape having smooth walls that form an enclosed passageway extending completely through the strut.

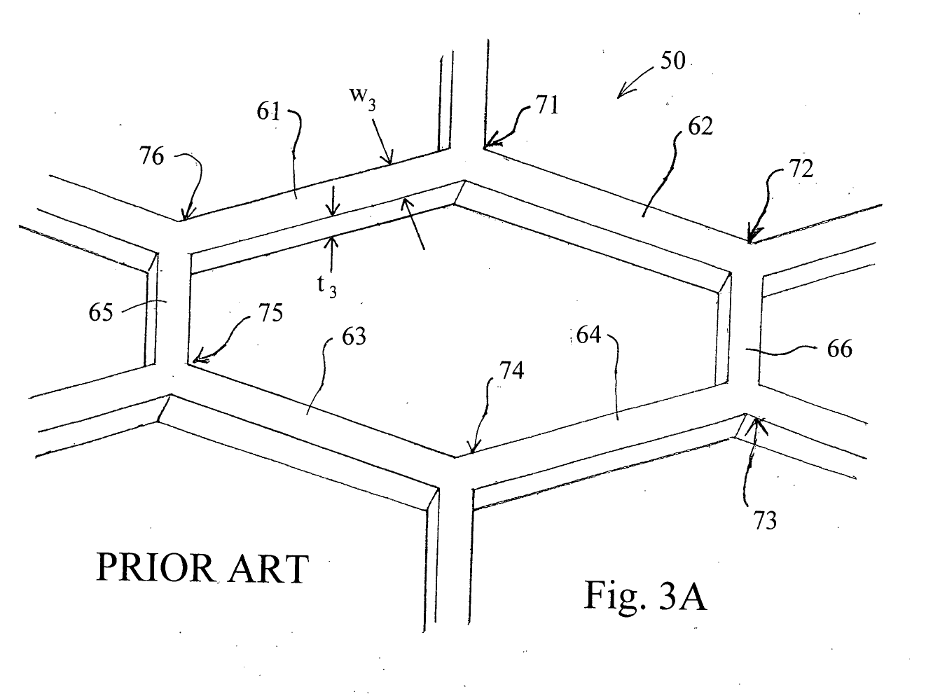

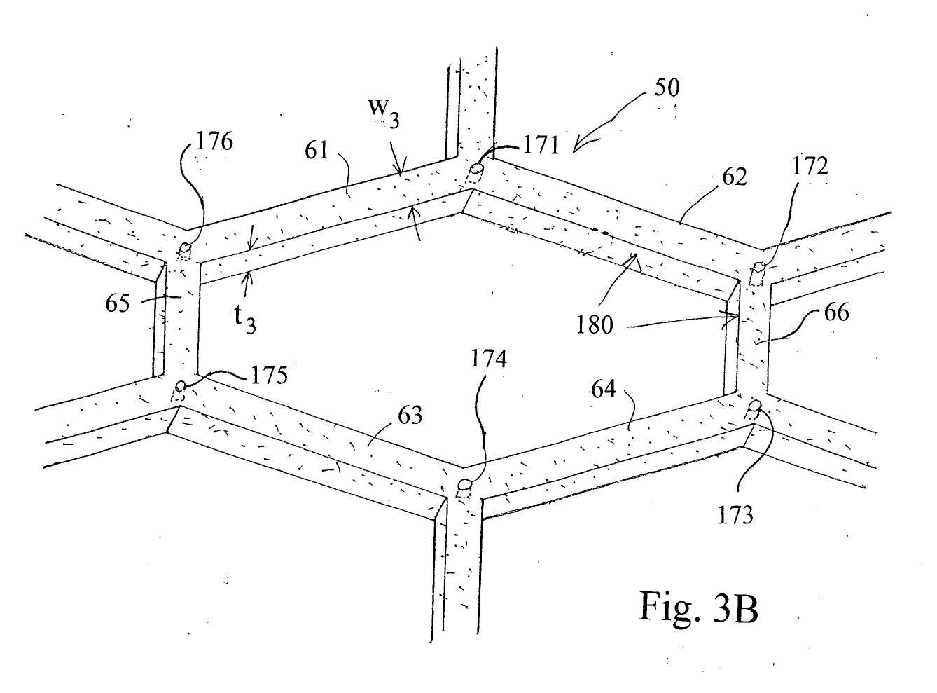

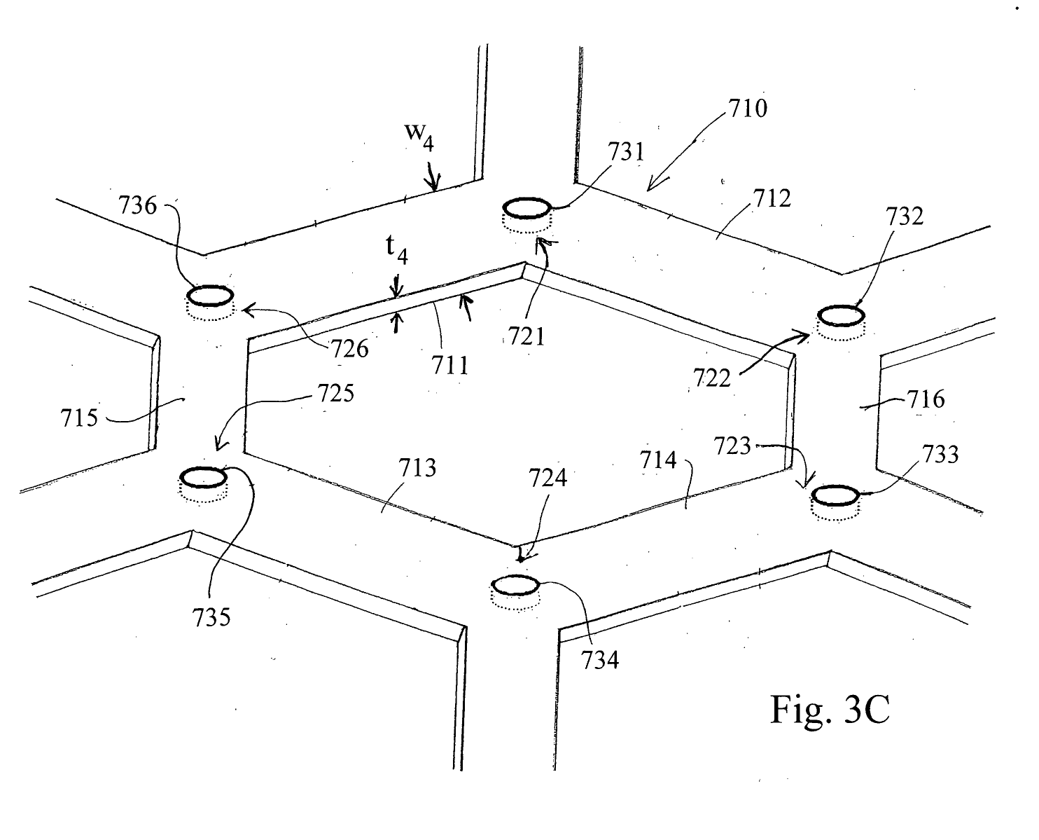

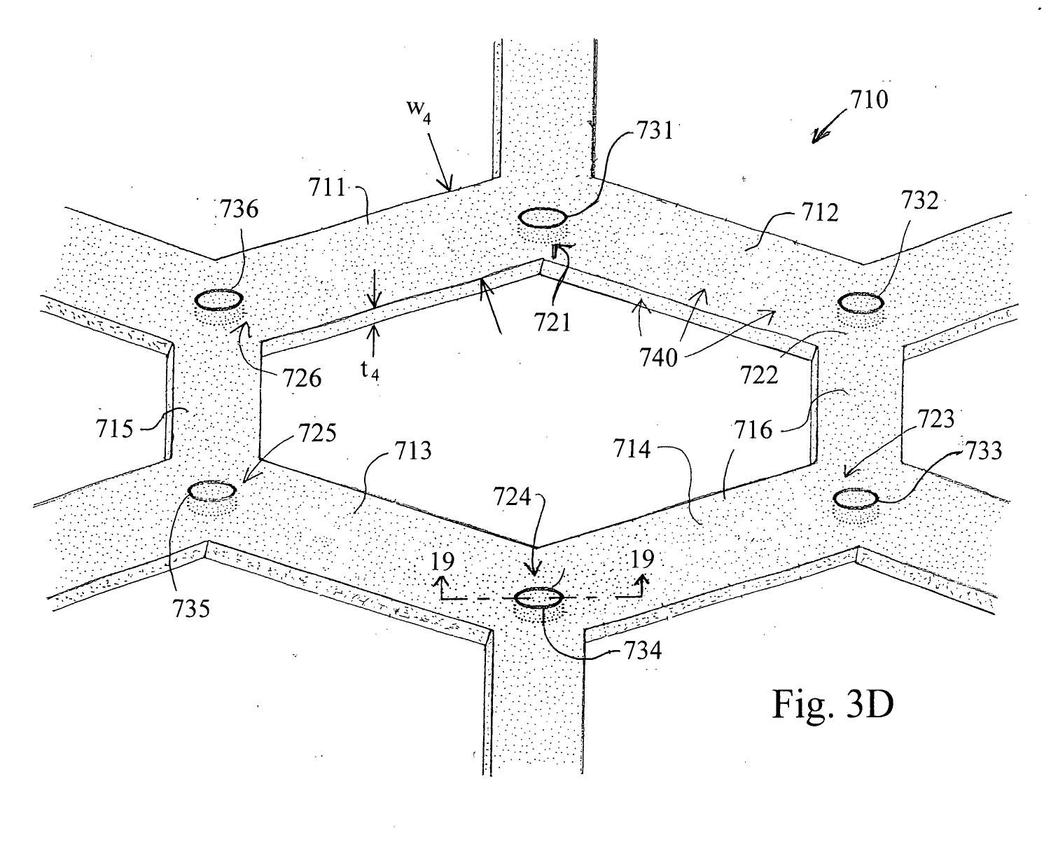

FIG. 3A illustrates another prior art stent cell configuration wherein the cell shown generally as 50 includes six struts 61-66 and six intersection points or flexion points 71-76 between adjacent struts. The cell configuration shown in FIG. 3A is taught by Palmaz U.S. Pat. No. 4,739,762. Each of the struts 61-66 has a rectangular cross section with a width w₃and a thickness t_{3.}

FIG. 3B illustrates how the relief holes of the present invention can be applied to the prior art cell configuration 50 of FIG. 3A to increase the flexibility of the cell configuration 50. As shown in FIG. 3B, a series of relief holes 171-176 has been formed by laser cutting at each of the flexion points 71-76 shown in FIG. 3A. Each of the relief holes 171-176 has a circular cross section and has a diameter that is between 30% and 70% of the width w₃ of each strut. Each of the relief holes 171-176 is formed along the center line of the struts and is spaced equally from the edges of the struts. The embodiment shown in FIG. 3B utilizes a single relief hole at each flexion point of cell 50, whereas the embodiment shown in FIGS. 2A and 2B utilizes three relief holes at the selected flexion points 118 and 119.

FIG. 3B illustrates the application of an optional medicinal coating 180 to the surface of each of the struts 61-65. The medicinal coating 180 has also been applied to and fills each of the relief holes 171-176.

FIG. 3C illustrates a cell configuration 710 that has an overall shape similar to the prior art cell configuration 50 shown in FIGS. 3A and 3B but wherein each of the struts 711-716 has a width W₄ substantially greater than the width w₃ and a thickness t₄ that is significantly less than the thickness t₃ of the prior art strut configuration shown in FIGS. 3A and 3B. As shown in FIG. 3C, at the intersections 721-726 of two or more struts, also referred to herein and in the claims as “flexion points,” an array of relief holes 731-736 have been formed as by laser cutting. As noted above, each of the relief holes 231-236 is placed along the center line of each strut and is spaced equidistantly from the edges of the struts. Each of the relief holes 731-736 is circular in cross section and has a diameter of between 30% and 70% of the width w₄ of each strut.

FIG. 3D illustrates the stent cell 710 shown in FIG. 3C after a medicinal coating 740 has been applied to all surfaces of the stent. In addition, the medicinal coating has been utilized to completely fill the relief holes 731-736.

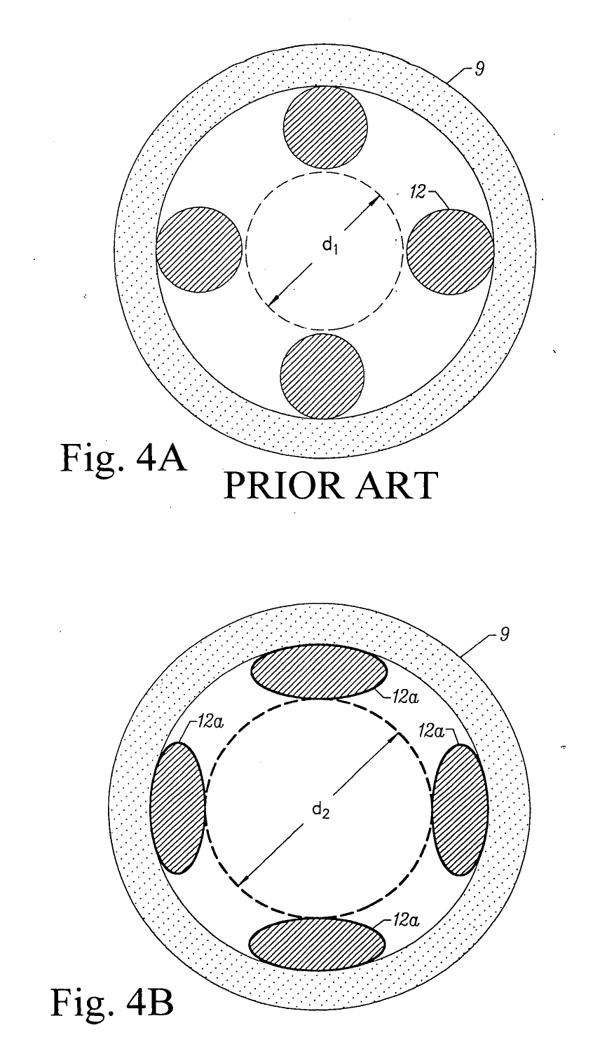

FIGS. 4A and 4B illustrate how the use of the invention with thinner and wider struts increases the capacity of a given artery for laminar, unobstructed blood flow. FIG. 4A shows artery 9 and a stent having struts 12 with circular cross section. The central, unobstructed region of artery 9 capable of laminar blood flow is shown by the dotted line having a diameter d₁. FIG. 4B shows the same artery 9 with a strut 12a of the present invention having reduced thickness and increased width compared with prior art strut 12. The central, unobstructed portion of artery 9 is shown by the dotted line having a diameter d₂. Any increase in this diameter is very significant, particularly as smaller diameter or diseased vessels are considered. The effective volume of less turbulent laminar blood flow varies with the square of the diameter d₂ so that the difference illustrated between FIGS. 4A and 4B represents an approximately 50% increase in theoretical volumetric low turbulence, laminar blood flow in the stented artery for the same degree of expansion. Reduction of turbulence provides the added benefit of reduced blood clotting, since turbulent blood flow tends to create blood clots. As noted above, the increased width of the stent members increases contact with the vessel wall as illustrated in FIG. 4B.

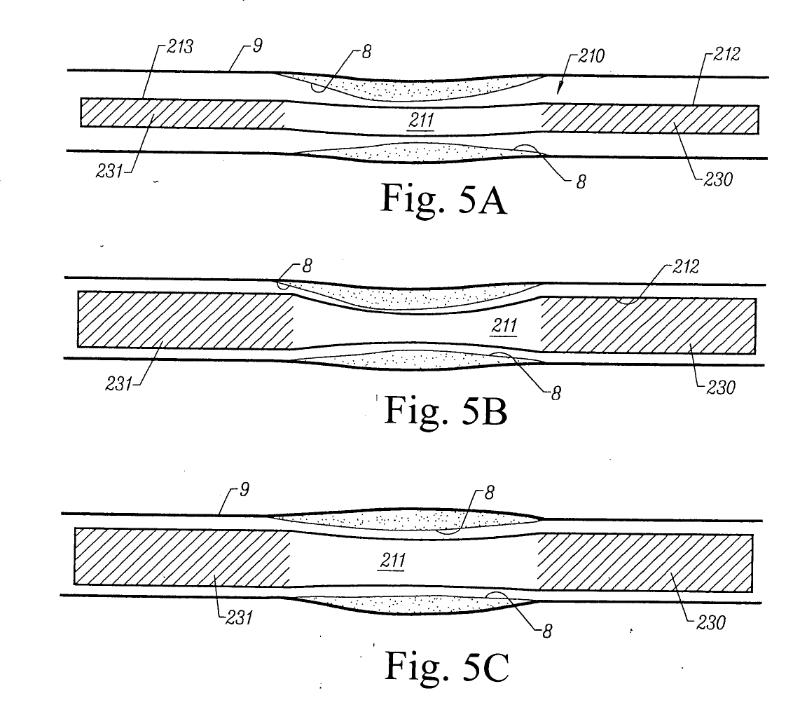

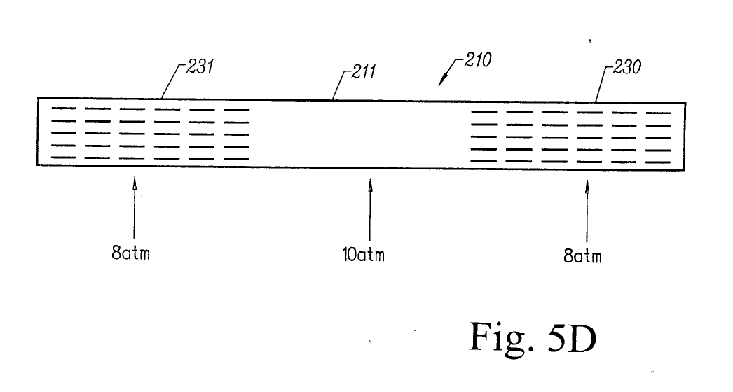

FIGS. 5A-5D illustrate how the present invention may be utilized to cause a stent to expand at its distal and proximal ends before it expands in its center region. A stent 210 is shown positioned in an artery 9 adjacent plaque deposit 8. The central region 211 of stent 210 is positioned adjacent the plaque deposit 8. The central region 211 of stent 210 has no relief holes formed therein. The distal region 212 of stent 210 has a plurality of relief holes shown generally as 230 formed therein. Similarly, the proximal end 213 of stent 210 has a plurality of relief holes 231 formed therein. For the sake of illustration purposes, the relief holes 230 and 231 in FIGS. 5A-5D are simply shown as cross hatching or dashed lines. It is to be understood that stent 210 may be any type of balloon expandable stent, including stent cell configurations illustrated in FIGS. 1-3 as well as a variety of other known stent configurations. For example, The Stenter's Notebook published by Physicians' Press in 1998 and written by Paul S. Phillips, M. D., Morton J. Kern, M. D. and Patrick W. Serruys, M. D. illustrates a variety of commercial, balloon expandable stents at pages 181-206. Those pages are herein incorporated by reference as though set forth in full herein. It is to be understood that the relief holes according to the present invention may be utilized in any of the balloon expandable stent designs illustrated in The Stenter's Notebook. Some of those stent designs do not use a plurality of closed cells, but rather use single wire shapes formed in a variety of ways. Each of those designs has the common feature that a portion of the stent is designed to flex or bend to allow the stent to expand. It is within the scope of the present invention to apply the relief holes of this invention to any of those prior art balloon expandable stent designs, preferably at the points of those stent designs where the maximum flexing and bending are designed to occur to allow the stent to expand. The invention may also be used in self-expanding stents, as discussed below.

The stent 210 illustrated generically in FIG. 5A is intended to include any of the stent configurations illustrated in The Stenter's Notebook. which are balloon expandable as well as those configurations illustrated in FIGS. 1-3 herein, and those shown and described in parent application Ser. No. 09/357,699, incorporated herein by reference as though set forth in full. With the relief holes formed at the distal and proximal ends 230 and 231, respectively, as the balloon (not shown for clarity) expands, the distal and proximal ends 230 and 231 expand first as illustrated in FIG. 5B. This feature can be very important since, as illustrated in FIG. 5B, the distal portion 230 and proximal portion 231 of the stent will contact the vessel wall 9 before the central region of the stent 211 contacts the plaque deposit 8, thereby tending to “trap” the plaque deposit in its present position. As illustrated in FIG. 5C, the central region 211 of the stent which does not have any relief holes requires somewhat additional pressure to expand and is shown in its expanded position where it contacts plaque deposit 8 and expands the restricted part of the artery. FIG. 5D illustrates typical balloon pressures; the distal and proximal ends 230,231 expand with 8 atm pressure and central section 211 expands at 10 atm pressure. Other expansion pressures can be used; FIG. 5D is presented only as an example.

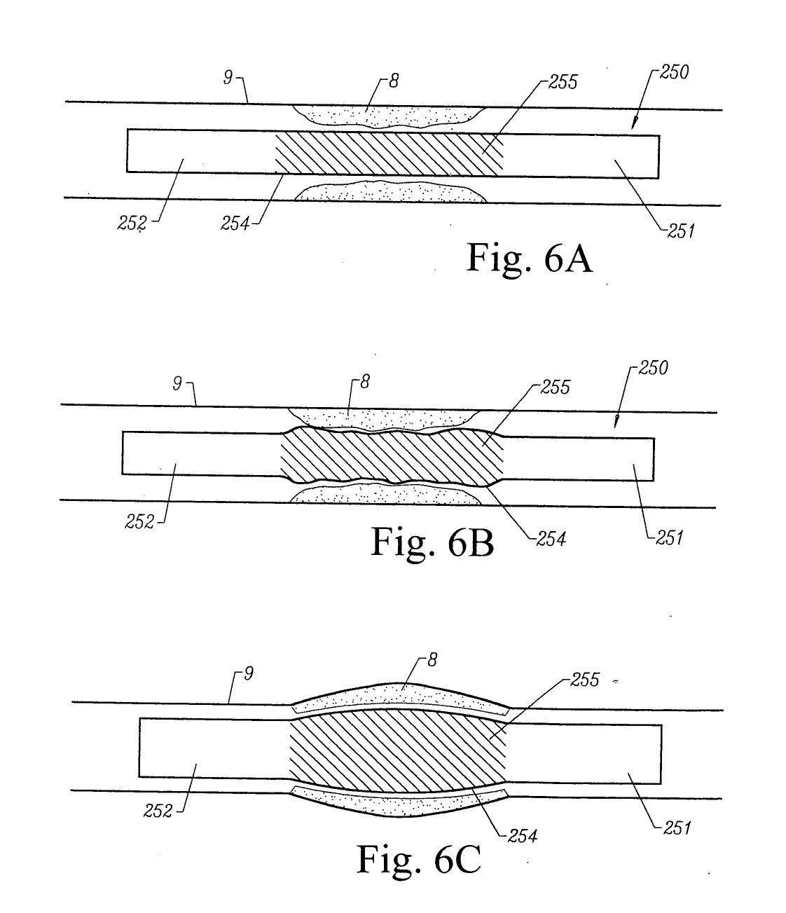

FIGS. 6A-6C illustrate a stent configuration 250 having no relief holes at its distal end 251 or at its proximal end 252. Stent 250 does have a plurality of relief holes 255 according to the present invention at its central region 254. As shown in FIG. 6B, the central region 254 having the relief holes expands first. This feature can be advantageous in preventing longitudinal motion of the stent relative to the artery as it expands.

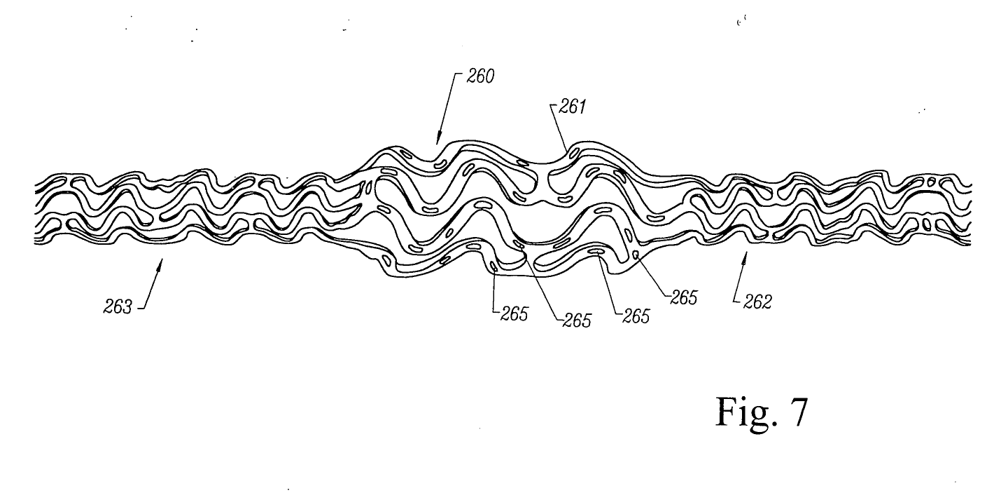

FIG. 7 illustrates yet another sinusoidal stent configuration 260 with relief holes 265 formed only in its central region 261. That central region 261 will expand prior to the distal and proximal regions 262 and 263, respectively.

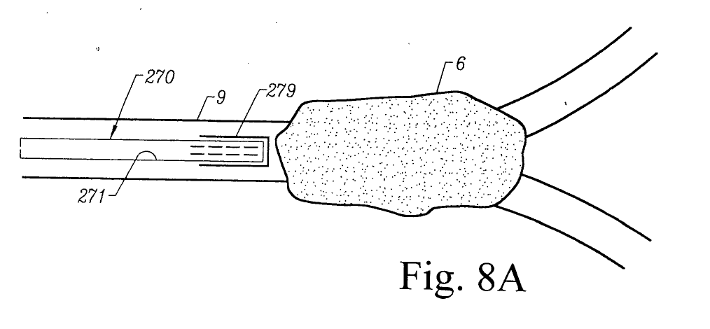

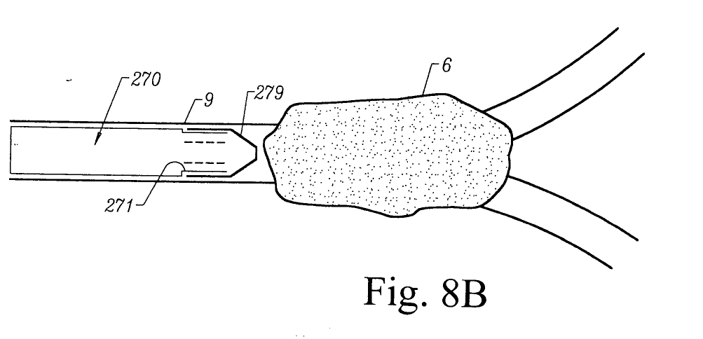

FIGS. 8A-8B illustrate how the present invention may be utilized to starve a cancerous tumor 6 fed by an artery 9. A stent 270 is placed in the artery close to the cancerous tumor 6. Stent 270 has relief holes formed only in its distal end 271. Stent 270 carries an impermeable covering sheath 279 and, as its distal end 271 expands and contacts the walls of artery 9, blood flow through artery 9 to tumor 6 is interrupted, causing the tumor to die.

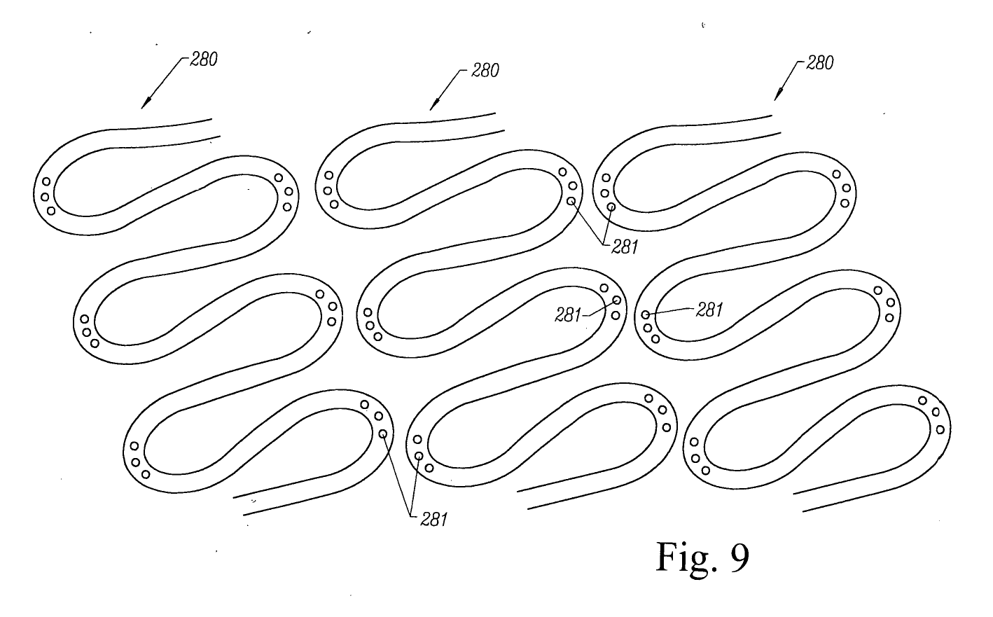

FIG. 9 illustrates how the present invention may be used with a continuous, single strand serpentine stent 280 known in the art. This type of stent is widely used in the art and is illustrated separately since it does not use a closed cell design but, nevertheless, may benefit significantly from the present invention. Relief holes 281 are formed in serpentine stent 280 at the intended points of maximum flex or bending as the stent is expanded. Using the relief holes as illustrated in FIG. 9 will allow the stent to expand in response to less balloon pressure. It is to be understood that the serpentine stent design could also be modified by increasing the width of the serpentine stent member and decreasing the thickness.

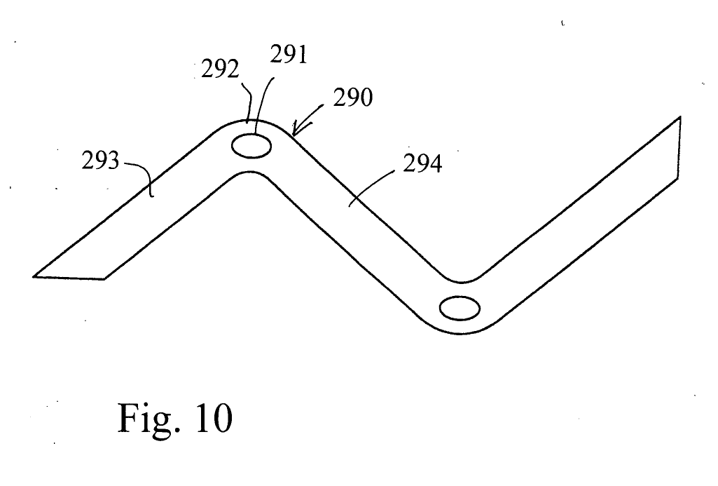

FIG. 10 illustrates another embodiment of the invention utilized in a continuous, serpentine stent 290. A single, arcuate relief hole 291 is formed at flexion point 292 between struts 293 and 294. Relief hole 291 may be elliptical, oval, circular or have a slotted design.

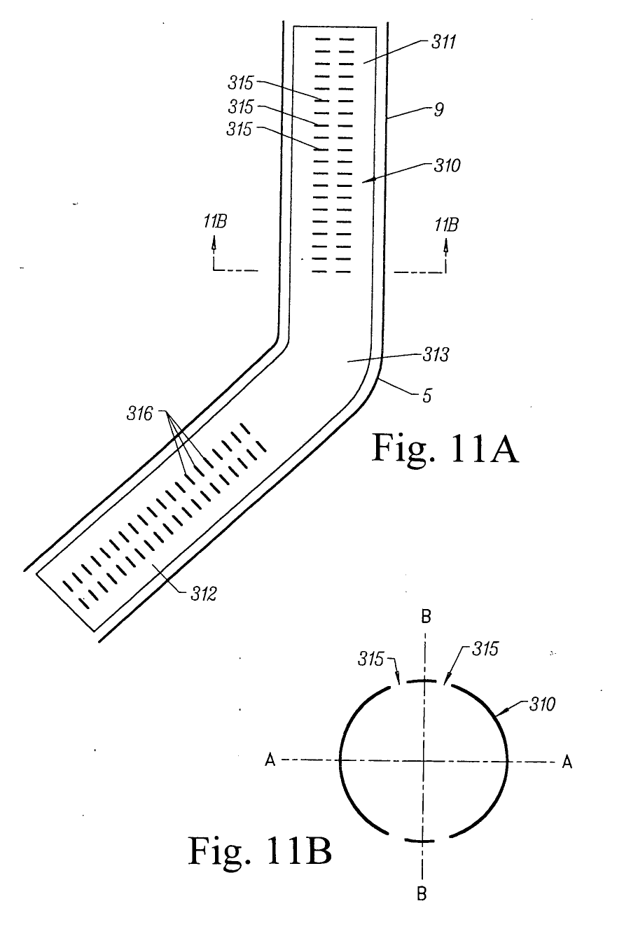

FIGS. 11-14 illustrate various techniques by which the relief holes of the present invention may be used advantageously in curved or angulated arteries or other body lumens. For example, FIG. 11A illustrates an artery 9 having a curved region 5. Artery 9 is assumed to lie in a plane parallel with the drawing. Stent 310 is shown positioned in artery 9 in its expanded position. Stent 310 has a distal section 311, a proximal section 312 and a central section 313. The distant and proximal sections each carries a pattern of relief holes illustrated by dashed lines 315 and 316, respectively. Central section 313 of stent 310 does not have relief holes formed in it in the embodiment illustrated in FIG. 11A. The relief holes 315 and 316 are not formed around the entire periphery of stent 310, as illustrated in sectional view 11B. As shown in sectional view 11B, relief holes 315 are formed in the upper part and lowermost part of stent 310; however, no relief holes are formed near the horizontal axis A illustrated in FIG. 11B. The effect of placing relief holes as illustrated in FIG. 11B is to allow stent 310 to bend easily relative to vertical axis B-B in order to accommodate the curved section 5 of artery 9 to facilitate deployment of stent 310. The pattern of relief holes as illustrated in FIGS. 11A and 11B tends to maximize the strength of stent 310 in its central region 313 to resist the crumpling of the curved region 5 of artery 9.

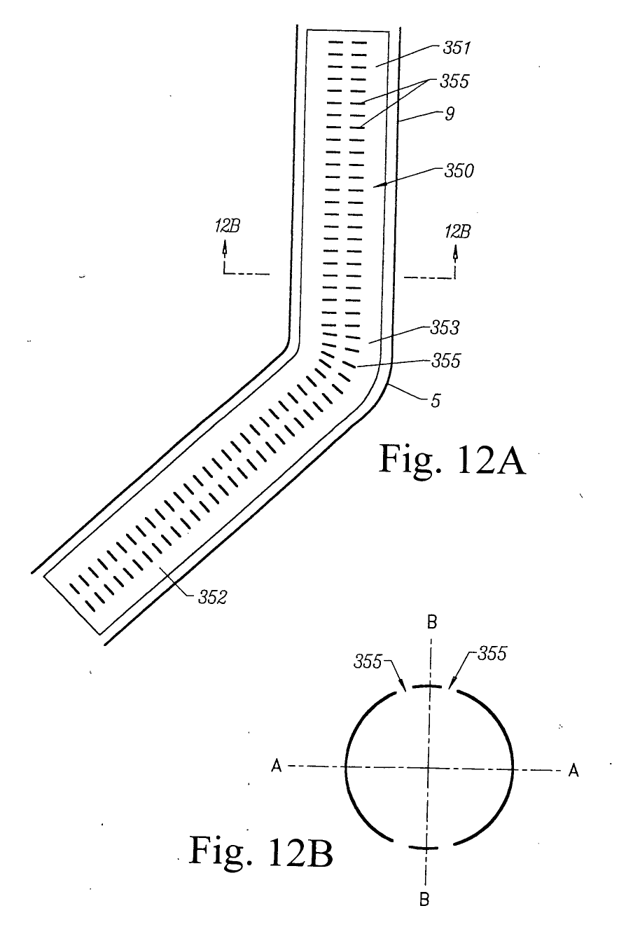

FIGS. 12A and 12B illustrate a variation to the relief hole pattern shown in FIG. 11. Stent 350 illustrated in FIG. 12 has relief holes 355 formed along its entire length including central region 353 as well as distal and proximal ends 351 and 352. Again, the relief holes 355 are only formed away from the horizontal axis A-A illustrated in FIG. 12B. Placement of the relief holes in this fashion allows the stent 350 maximum flexibility to bend about vertical axis B-B illustrated in FIG. 12B to facilitate its placement and deployment in the curved artery 9.

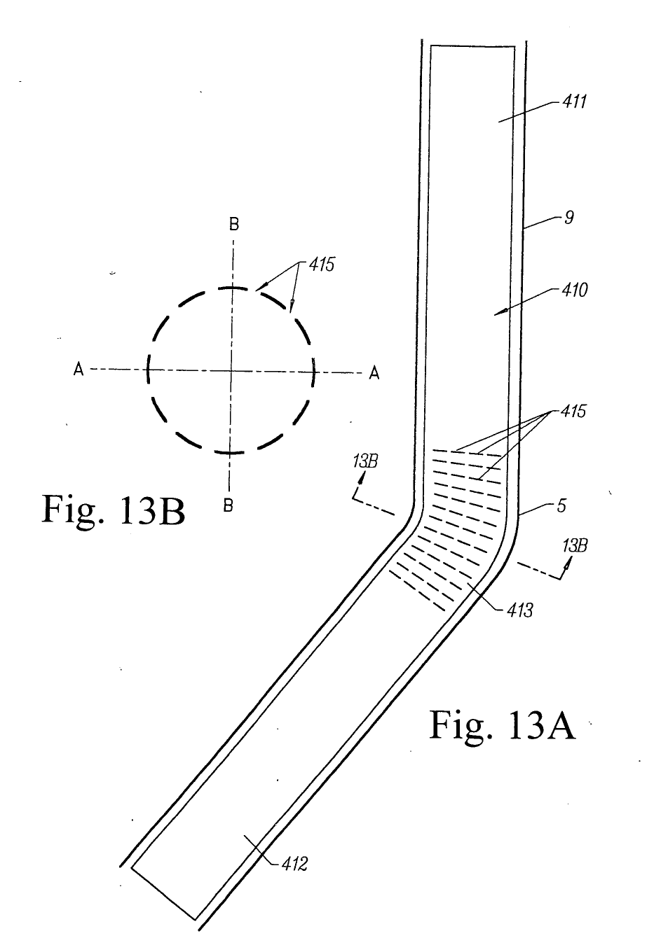

FIGS. 13A and 13B illustrate yet another manner in which relief holes may be utilized to facilitate deployment of stent 410 in curved artery 9 having a curved section 5. In this embodiment, the pattern of relief holes 415 is only formed in the central region 413 of stent 410. The proximal end and distal end 411,412 have no relief holes formed therein. Central section 413 has relief holes formed completely and uniformly around its periphery as illustrated in sectional view 13B. Placement of relief holes around the entire periphery of the central region 413 allows maximum flexibility of stent 410 in the region where stent 410 must bend to conform to the curved region 5 of artery 9.

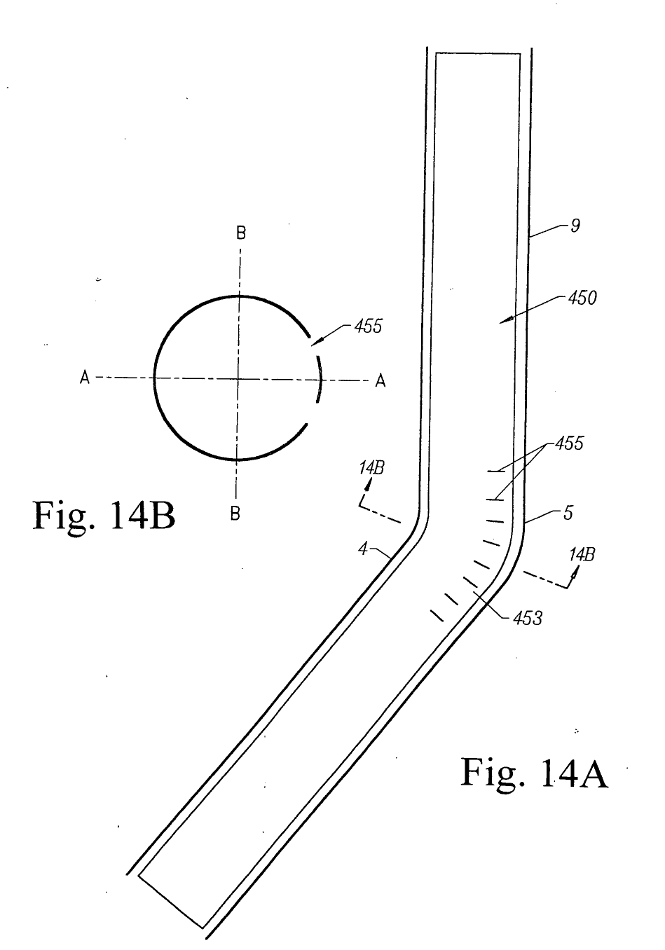

FIGS. 14A and 14B illustrate stent 450 having a pattern of relief holes 455 placed only at the central region 453 of stent 450 and only in that portion of the stent periphery which contacts the outside radius of the arterial wall at the curved section 5. As illustrated in FIG. 14B, relief holes 455 are placed radially outwardly of the vertical axis B-B of FIG. 14B and no relief holes are formed on the inside radius, that is, radially inwardly of the central vertical axis B-B of stent 450. The purpose of placing relief holes in this fashion is to allow the central portion 453 of stent 450 to flex to conform to the curved portion 5 of artery 9 while simultaneously allowing the stent to remain as rigid as possible adjacent the more sharply curved arterial wall region 4 which occurs at the radial inwardly side of the curved section of artery 9. The stent is therefore strongest along the inside radius 4 of curved artery 9, which is the part of the artery most likely to crimp.

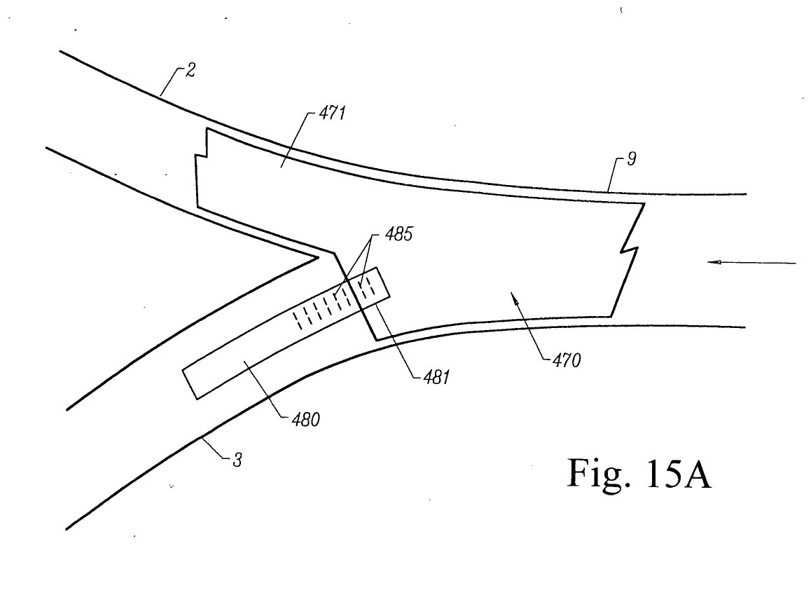

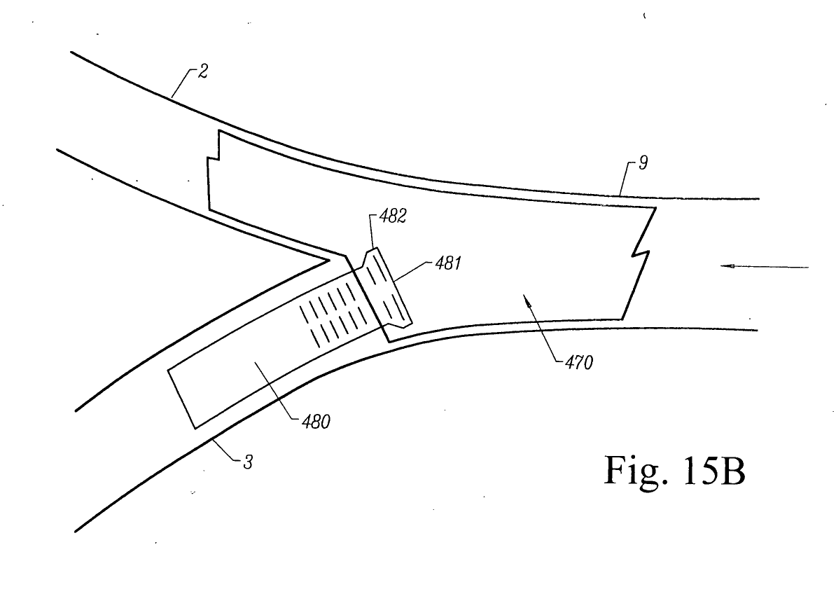

FIGS. 15A and 15B are schematic illustrations showing how the present invention can be used in conjunction with bifurcated stents. A bifurcated artery 9 splits into two branches 2 and 3. A first prior art stent 470 is placed in artery 9 and has an extension 471 that extends partially into branch 2. A second stent 480 is provided having a series of relief holes 485 formed in its distal end 481. The distal end 481 of stent 480 is positioned inside stent 470 prior to being expanded. As shown in FIG. 15B, as stent 480 is expanded, its distal end 481 forms a “flare” 482 which effectively seats against stent 470 and which prevents stents 470 and 480 from separating after being deployed.

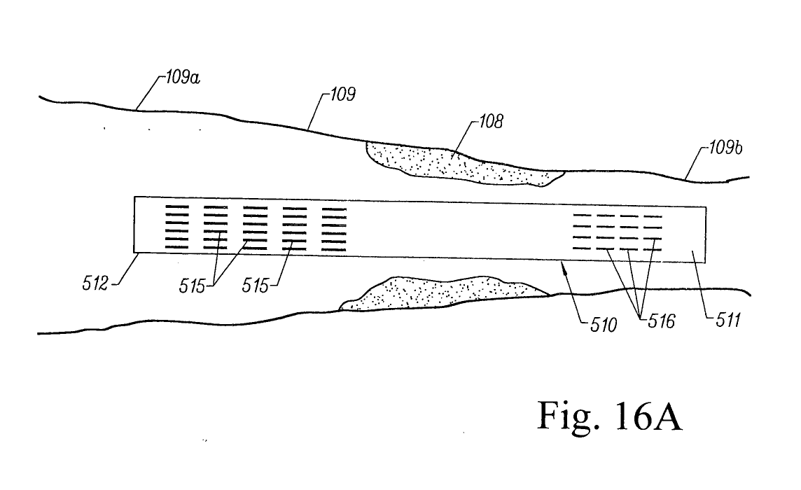

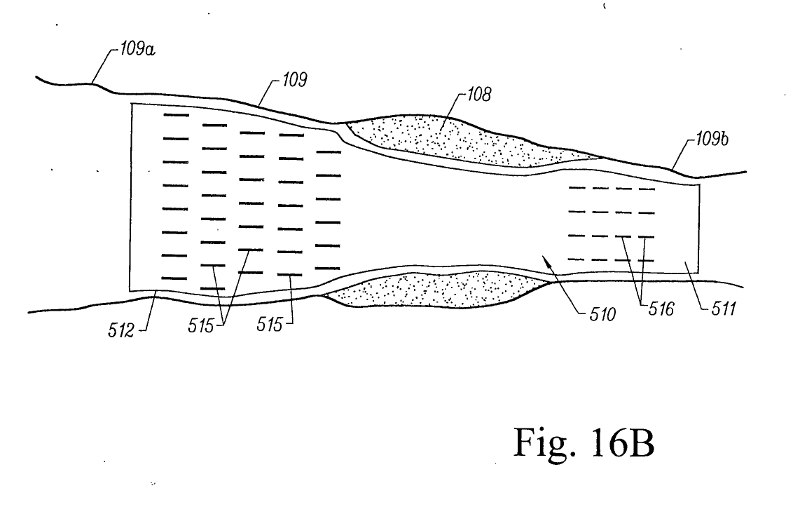

FIGS. 16A and 16B are schematic illustrations showing how the present invention can be utilized in arteries or other lumens having somewhat irregular shapes. Artery 109 is shown having a first section 109a of rather large diameter and a second section 109b having a somewhat reduced diameter. Plaque deposit 108 is illustrated in the generally tapered region of artery 109. A stent 510, which can be any prior art stent or any stent shown and described in the parent application referred to above. Stent 510 has a distal end 511 and a proximal end 512. A rather large number of relief holes 515 are formed in proximal end 512 of stent 510. A somewhat smaller number of relief cuts is formed in the distal end 511 of the stent 510. The purpose of placing these patterns of relief holes on stent 510 is to cause the stent 510 in its expanded position to conform as closely as possible to the walls of the artery 109 and the plaque deposit 108. As shown in FIG. 16B, the proximal end 512 expands further because of the presence of a greater number of relief holes 515. The distal end 511 expands a somewhat reduced amount because of the absence of relief holes. The small pattern of relief holes 516 formed near the distal end 511 causes a somewhat greater expansion of stent 510 in that region to conform to the shape of the arterial wall and plaque deposit 108. FIGS. 16A and 16B illustrate how patterns of relief holes can be utilized to make a stent expand in a controlled non-uniform fashion to conform to a somewhat irregularly shaped arterial wall. The stent in its expanded form effectively supports the irregularly shaped vessel wall and plaque deposits and simultaneously seals off any leaks that would otherwise occur at the proximal and distal ends of the stent.

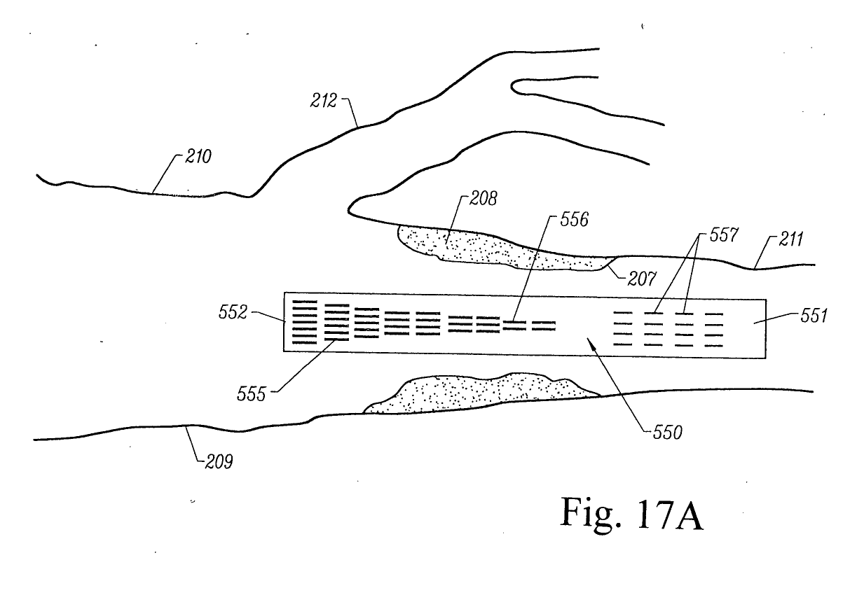

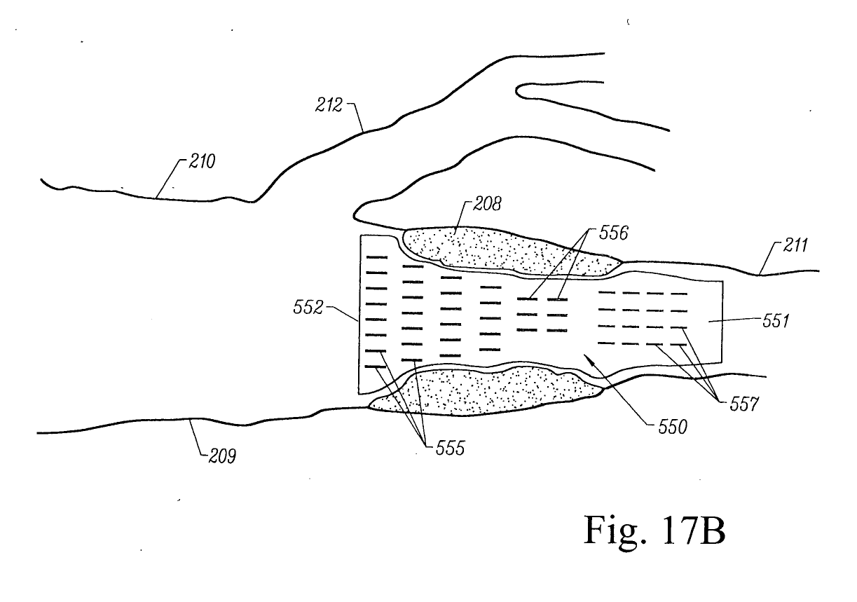

FIGS. 17A and 17B are schematic illustrations showing how the present invention may be utilized in an artery or other lumen having a branch artery at or near the location where the stent is to be deployed. Artery 209 has a first region 210 of relatively large diameter and a second downstream region 211 having a considerably reduced diameter. A branch artery 212 connects to artery 209 near a plaque deposit 208. Stent 550 is provided having a distal end 551 and a proximal end 552. Since the proximal end 552 must expand a greater distance than the distal end 551, a relatively large number of relief holes 555 is placed near proximal end 552. The number of relief holes is gradually reduced and, at the center of stent 550, a relatively sparse pattern 556 of relief cuts is applied where the stent should be expanded the least. Towards the distal end of stent 551 a secondary pattern 557 of relief holes is applied so that the stent may expand to a somewhat greater degree adjacent the distal end 207 of plaque deposit 208. Stent 550 is shown in its expanded form in FIG. 17B and it can be seen that the patterning of relief holes allows the stent to expand in a controlled non-uniform fashion to conform to the walls of the artery 209 and to achieve the desired blood flow through artery 209.

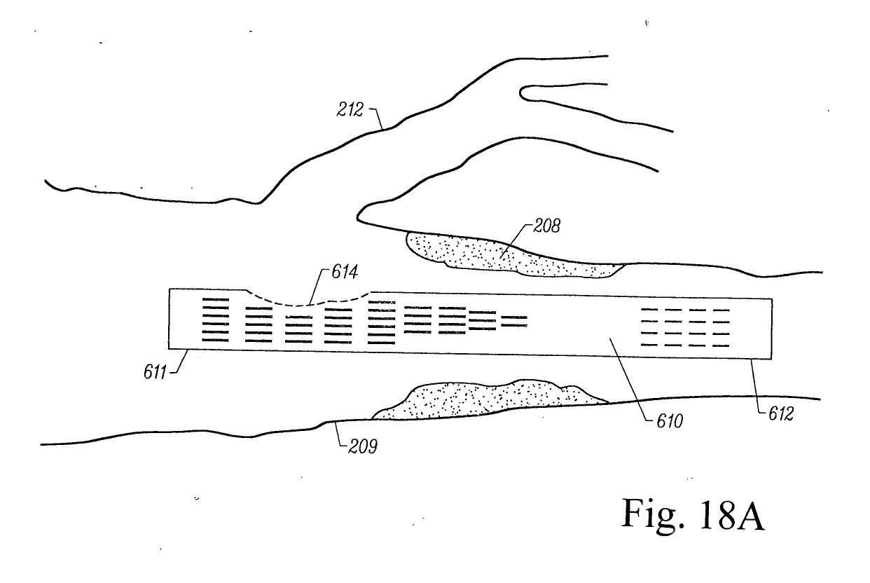

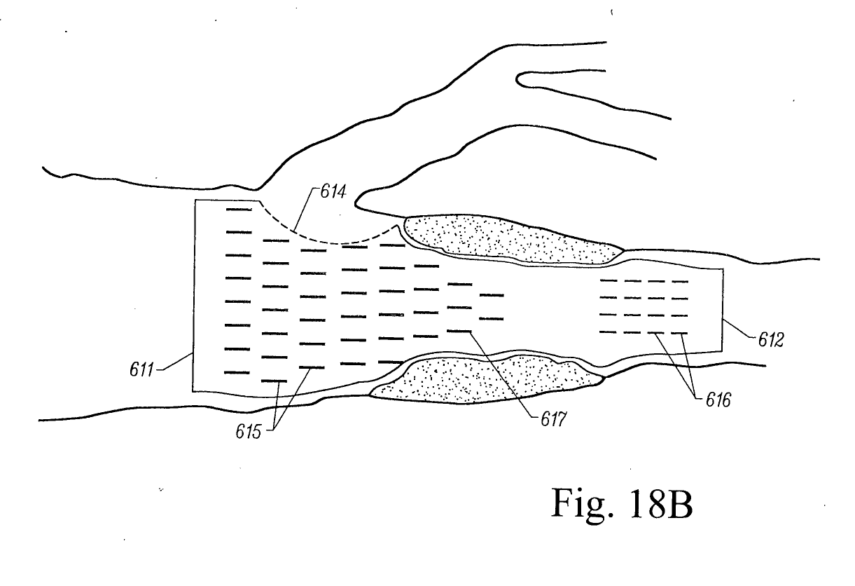

FIGS. 18A and 18B show an alternate stent design 610 which may be utilized in the irregular shaped artery 209 illustrated in FIGS. 17A and 17B with plaque deposit 208 and branch artery 212. Stent 610 has a proximal end 611 and a distal end 612. The proximal end of stent 610 extends beyond the location where branch artery 212 connects with artery 209. Stent 610 has an opening 614 formed in its surface adjacent where stent 610 will expand against the base of branch artery 212. The opening 614 in stent 610 allows blood to flow freely from artery 209 into branch artery 212. Stent 610 has a greater number of relief holes 615 formed at its proximal end 611 as compared to the relief holes 616 formed at its distal end. A tapering pattern of relief holes 617 is formed in the center of stent 610 to allow the stent to conform to the required taper. As shown in FIG. 18B, the relief hole patterns are designed to allow the stent to expand in a controlled, non-uniform manner to conform to the wall of the artery (or other lumen) and to prevent end leaks.

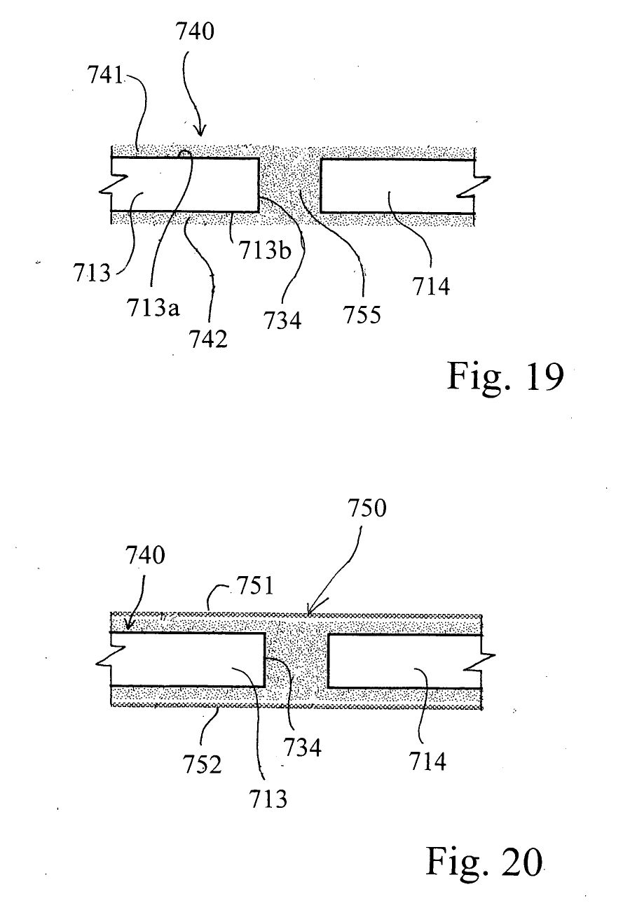

FIG. 19 is a sectional view showing relief hole 734 formed at the flexion point between struts 713 and 714 as shown in FIG. 3D. The medicinal coating 740 is shown having an upper layer 741 covering the upper (or outer) surface 713a of strut 713 and a lower layer 742 that covers the lower (or inner) surface 713b of strut 713. The upper layer 741 and lower layer 742 are connected by a “plug” of material 755 that fills the relief hole 734. The upper layer 741 forms the outer layer when the cylindrical stent expands, and lower layer 742 forms an inner layer when the stent expands. The plug 755 connects the upper surface 741 and lower surface 742 and greatly enhances the adhesion of the coating 740 to each individual strut, such as strut 713. The presence of relief holes, particularly at or near the flexion points of the struts subjected to the most flexion and bending during expansion of the stent, significantly reduces the likelihood of the coating separating from the surface of the struts as the stent is expanded. Furthermore, the “plug” 755 provides additional material to dissolve into the vessel wall. If coating 740 is a dissolvable medicine, after the outer surface 741 dissolves, plug 755 dissolves and extends the time period during which medicine is applied. It is also within the scope of the invention to apply the coating by spraying the outer surface of the stent, and allow the sprayed coating to extend into and through the relief holes, without coating the inner surface of the stent.

FIG. 20 illustrates an alternate embodiment of the invention wherein a second coating 750 is applied directly on top of first coating 740 (see FIG. 19). Second coating 750 may also be applied to dipping the stent so that the second coating 750 has an upper (or outer) layer 751 which covers the upper (or outer) layer 741 of coating 740 and a lower (or inner) layer 752 that completely covers the lower (or inner) layer 742 of coating 740. Both coatings 740 and 750 may be medicinal coatings. Alternately, coating 740 could be primarily an adhesive coating to further increase the adhesion of coating 750 to the stent struts and which is also particularly adapted to form a tight adhesive bond with second coating 750, which may be a particular medicinal coating that does not bond well if applied directly to the material which comprises the stent strut.

Although the above description of the invention has concentrated on balloon expandable stents, the invention is also useful with self-expanding stents. In the case of self-expanding stents, the use of relief cuts allows the use of flatter and thinner walled stents, increasing the radio-opacity and vessel wall coverage of the stent. Furthermore, using patterns of relief cuts in self-expanding stents, those stents may be caused to expand in a controlled, non-uniform fashion which can be advantageous in many situations. Each of the figures illustrated herein, including the various stent cell configurations and the various patterns of relief cuts illustrated in the drawings, may all be applied to self-expanding stents.

The present invention is usable with stents made of any solid, non-tubular, non-porous material such as nitinol, stainless steel, plastic and composite materials.

The foregoing description of the invention has been presented for purposes of illustration and description and is not intended to be exhaustive or to limit the invention to the precise form disclosed. Many modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is defined by the following claims.

1. An expandable stent, comprising:
a plurality of interconnected solid struts, each of said struts having a centerline and a cross-sectional width and thickness, wherein said width is between 1.5 and 5 times as great as said thickness,
a plurality of junctures formed where two or more of said struts are interconnected, said junctures acting as flexion points about which said struts move as said stent is expanded,
each of said struts having a width at each of said plurality of junctures the same as the width of said strut between junctures, and
an array of arcuate relief holes formed in some of said junctures, wherein each of said arcuate relief holes is located along the centerline of each strut and equidistantly from the edges of said strut and wherein each of said arcuate relief holes extends completely through said strut,
each of said arcuate relief holes having a width that is between 30% and 70% of the width of said strut, and each of said arcuate relief holes having a length along said centerline that is less than the width of said strut,
whereby as said stent expands, said struts flex elastically at said junctures and wherein the presence of said arcuate relief holes allows said stent to expand more easily than without said relief holes.

2. The stent of claim 1 wherein said stent has a designed range of expansion and wherein the expansion of said stent through the designed range of expansion occurs without plastic deformation of said struts either at said flexion points or between flexion points.

3. The apparatus of claim 1 wherein said stent has distal and proximal ends and a central section, and wherein said arcuate relief holes are formed only in said distal and proximal ends.

4. The apparatus of claim 1 wherein said stent has distal and proximal ends and a central section, and wherein said arcuate relief holes are formed only in said central section.

5. The apparatus of claim 1 wherein said arcuate relief holes are applied to said stent in patterns to allow controlled, non-uniform expansion of said stent.

6. The apparatus of claim 1 wherein said arcuate relief holes are circular.

7. The apparatus of claim 1 wherein said arcuate relief holes are elliptical.

8. The apparatus of claim 1 further comprising a coating applied to said stent wherein said coating extends into at least some of said arcuate relief holes.

9. The apparatus of claim 8 wherein said coating is a medicinal coating.

10. The apparatus of claim 9 wherein said medicinal coating fills some of said arcuate relief holes and forms plugs of medicinal material in said relief holes.

11. The apparatus of claim 8 wherein said coating comprises first and second separate layers, said first layer applied to said inner and outer surfaces of said stent, and said second layer being formed on top of said first layer.
